(19) United States
(12) Patent Application Publication
(10) Pub. No.: US 2010/0021975 A1
(43) Pub. Date: Jan. 28, 2010

(54) Process For Producing Xylo-Oligosaccharides

(75) Inventors: Herbert Sixta, Lenzing (AT); Roland Möslinger, Zell am Pettenfirst (AT); Thomas Lange, Vöcklabruck (DE)

Correspondence Address: FITZPATRICK CELLA HARPER & SCINTO, 1290 Avenue of the Americas, NEW YORK, NY, 10104-3800, US

(73) Assignee: LENZING AKTIENGESELLSCHAFT, Lenzing (AT)

(21) Appl. No.: 12/299,981

(22) PCT Filed: May 9, 2007
(86) PCT No.: PCT/AT2007/000223

§ 371 Date: Jun. 10, 2009

(30) Foreign Application Priority Data
May 10, 2006 (AT) A 808/2006

(51) Int. Cl. C12P 19/04; C07H 1/06

(52) U.S. Cl. 435/101; 536/127

(57) ABSTRACT

The invention relates to a process for producing xylo-oligosaccharides, comprising the steps of cold caustic extraction (CCE) of a pulp obtained by a cooking process; purifying at least a portion of the press liquor forming in the cold caustic extraction stage using a membrane separation process, preferably nano- or ultrafiltration; obtaining the xylans from at least a portion of the retentate of the membrane separation process; processing at least a portion of the xylans obtained into xylo-oligosaccharides.

### BACKGROUND OF THE INVENTION

1. Field of the Invention

The present invention relates to a process for producing xylo-oligosaccharides.

2. Description of the Related Art

It is known that stock flows accumulating during the production of pulps contain an amount of hemicelluloses which is sometimes substantial.

In WO 2005/118923, for example, the purification of the press liquor of a cold extraction process (a CCE process) using a membrane separation process is described. The retentate accumulating thereby contains the entire amount of the beta-cellulose present in the press liquor and more than half of the gamma-cellulose. In case of hardwood pulps, the beta-cellulose solution consists of almost pure xylan.

It is known per se to obtain hemicelluloses, e.g., xylans, from stock flows of the pulp production process. In most cases, the hemicelluloses thus obtained are used and/or resold, respectively, as such.

In contrast, the present invention relates to a process for obtaining xylo-oligosaccharides, i.e., xylan chains degraded to a low degree of polymerization of typically 2-10.

### SUMMARY OF THE INVENTION

The process according to the invention for producing xylo-oligosaccharides comprises the steps of

cold caustic extraction (CCE) of a pulp obtained by a cooking process

purifying at least a portion of the press liquor forming in the cold caustic extraction stage using a membrane separation process, preferably nano- or ultrafiltration

obtaining the xylans from at least a portion of the retentate of the membrane separation process

processing at least a portion of the xylans obtained into xylo-oligosaccharides

Thus, the process according to the invention utilizes the xylans obtained from the press liquor of a CCE process in order to produce xylo-oligosaccharides therefrom. Xylo-oligosaccharides have various applicabilities, e.g., as a food additive with prebiotic effect.

For producing the xylo-oligosaccharides from the xylans obtained, a process selected from the group consisting of hydrothermolysis and enzymatic hydrolysis may preferably be carried out.

The membrane separation process for concentrating the xylans in the press liquor of the CCE process may be carried out in two stages, with a dilution of the retentate of the first stage with water being performed between the two separation stages.

In doing so, a nanofiltration of the diluted retentate or also a dialysis of the retentate of the first stage may be carried out in the second stage. In both cases, the NaOH recovery is increased by said step, with the hemicellulose phase being concentrated at the same time.

In US 2005/0203291, a process for obtaining xylans from a phase rich in xylans is described.

In contrast to said known process, in a preferred embodiment of the process according to the invention, the xylans are obtained from the retentate of the membrane separation process by reverse precipitation.

For the reverse precipitation, an agent from the group consisting of mineral acids, CO₂ and monovalent or polyvalent alcohols is preferably used. Subsequently, the sedimentation residue can be washed in suitable installations (e.g., a washing press) and dehydrated mechanically.

### BRIEF DESCRIPTION OF THE DRAWING

Below, the present invention is explained in further detail by the figure and the exemplary embodiment.

FIG. 1 shows the molar mass distribution of a xylan isolated in mineral acid from a CCE filtrate by reverse precipitation.

### DETAILED DESCRIPTION OF THE INVENTION

### Example 1

### Isolation of Xylan from the CCE Filtrate

With the aid of a pilot nanofiltration plant (NF) equipped with a polyethersulfone membrane, Nadir N30 F, cut-off 300, a filtrate of a CCE stage was prefiltered using a cartridge filter (Profile Star AB I A4007J) in order to filter out remaining fibres and undissolved particles.

The NF plant was operated at a temperature of 40° C., a pressure of 25 bar and a specific flow rate of 5 l/m².h. The beta-cellulose (xylan) concentration in the feed amounted to approx. 15 g/l, the NaOH concentration was 90 g/l. The ratio of flow and hence also the liquor loads in the permeate to the retentate were 0.82:0.18.

Due to the low cut-off of the membrane, the total amount of beta cellulose in the retentate was at a concentration of about 85 g/l. The retentate was diluted with water at a ratio of 1:1.5 and again was supplied to the NF.

The NF conditions of the second stage were comparable to those of the first stage, with the exception that the amount of permeate dropped to about 70% of the feed amount. In the retentate of the second NF stage, the beta cellulose was now present at a concentration of about 115 g/l and at a NaOH concentration of about 32 g/l (hemi liquor).

Said substrate served for the isolation of the beta-cellulose (xylan) by reverse precipitation with mineral acid. For this purpose, approximately 0.1 parts of a sulfuric acid diluted 1:3 with water were mixed with 1 part of hemi liquor, whereby a pH of between 4 and 5 appeared in the end.

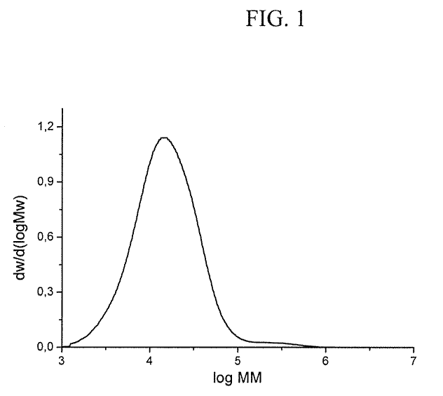

After about 6-8 h, an off-white precipitate with a consistency of approx. 10-15% by weight forms. Said precipitate was then centrifuged, washed and dried. A xylan content of 74% by weight was detected in this solid substance. Based on the total carbohydrate content, the xylan content amounted to 97.5%. The molar mass distribution of said xylan powder as determined by GPC shows a weight-average molecular mass of 24.4 kg/mol and a number-average molecular mass of 10.6 kg/mol (see FIG. 1).

### Example 2

### Further Processing of the Xylan Powder into Xylo-Oligosaccharides (XOS)

XOS was produced from the xylan in two ways, (A) hydrothermolytically, (B) enzymatically:

Hydrothermolytic XOS production:
318 g of dry xylan was diluted in 9.08 L water (xylan concentration 35 g/L) and was treated in an 11 L-pressure reactor at 120° C. for 5 h under continuous circulation. The circulation flow rate was 75 L/h. Upon completion of the reaction, the pH value of the hydrolysate was 3.13.

Subsequently, the solution was neutralized with 0.3 N NaOH to pH 6.5-7.0. The water-soluble products were spun down at 4000 U/min for 60 min. The isolated amount of XOS was 189.7 g of a freeze-dried powder, corresponding to a yield of 59.6%, based on the xylan used. The XOS mixture produced hydrothermolytically exhibits a relatively uniform composition across the examined DP-range (see bottom part of Table 1).

### Enzymatic Hydrolysis:

XOS₂₋₁₀

90 g of wet xylan (equal to 40 g of dry xylan) was suspended in 900 mL water (44.4 g/L) and 50 mg Pentopan Mono BG (1.25 mg/g xylan) was added. The mixture was stirred in a fermenter at 50° C. for 2 h. Thereupon, the enzyme was inactivated by 10 minutes of heating to 99° C. The remaining insoluble portions were spun down, the aqueous solution was freeze-dried. The yield of XOS, based on the xylan used, amounted to 75%.

XOS₂

The reaction was carried out in a 7L-fermenter with a filling of 6 L, the xylan concentration amounted to 20 g/L (120 g xylan). The production was likewise effected with the enzyme Pentopan Mono BG, but with an increased concentration of 1 g/L (50 mg/g xylan). Fermentation was carried out at 40° C. for 96 h. Reprocessing occurred as described above. The yield of XOS amounted to 78%, based on the amount of xylan used (93.66 g XOS). The product consists primarily of xylobiose (Table 1).

TABLE 1
Relative mass fractions of individual neutral XOS

| | |
|---|---|
| Hydro- | Enzymatically |

| Product | thermally | XOS₂₋₁₀ | XOS₂ |
|---|---|---|---|
| distribution | % by weight | | |
| X1 | 11.2 | 0.1 | 4.7 |
| X2 | 9.7 | 13.0 | 83.1 |
| X3 | 10.6 | 21.0 | 12.2 |
| X4 | 12.2 | 16.0 | |
| X5 | 12.5 | 11.0 | |
| X6 | 12.0 | 7.9 | |
| X7 | 12.6 | 7.2 | |
| X8 | 10.3 | 7.2 | |
| X9 | 9.0 | 6.9 | |
| X10 | | 9.7 | |
| XOS | 100.0 | 100.1 | 100.0 |

What is claimed is:

1. A process for producing xylo-oligosaccharides, comprising the steps of:
cold caustic extraction (CCE) of a pulp obtained by a cooking process
purifying at least a portion of the press liquor forming in the cold caustic extraction stage using a membrane separation process, preferably nano- or ultrafiltration
obtaining the xylans from at least a portion of the retentate of the membrane separation process
processing at least a portion of the xylans obtained into xylo-oligosaccharides

2. The process according to claim 1, wherein, for producing the xylo-oligosaccharides, a process selected from the group consisting of hydrothermolysis and enzymatic hydrolysis is carried out.

3. The process according to claim 1 or 2, wherein the membrane separation process for purifying the press liquor is carried out in two stages, with a dilution of the retentate of the first stage with water being performed between the two separation stages.

4. The process according to any of claims 1 to 3, wherein the xylans are obtained from the retentate of the membrane separation process by reverse precipitation.

5. The process according to claim 4, wherein, for the reverse precipitation, an agent selected from the group consisting of mineral acids, CO₂ and monovalent or polyvalent alcohols is used.
